(19) United States
(12) Patent Application Publication
(10) Pub. No.: US 2003/0114358 A1
(43) Pub. Date: Jun. 19, 2003

(54) Use of compatible solutes as inhibitors of the enzymatic decomposition of macromolecular biopolymers

(76) Inventors: Erwin Galinski, Senden (DE); Michael Kaufmann, Herdecke (DE); Thomas Schwarz, Leichlingen (DE); Maya-Devi Vangala, Schwerte (DE)

Correspondence Address: JACOBSON HOLMAN PLLC, 400 SEVENTH STREET N.W., SUITE 600, WASHINGTON, DC, 20004, US

(21) Appl. No.: 10/182,740

(22) PCT Filed: Feb. 14, 2001
(86) PCT No.: PCT/EP01/01611

(30) Foreign Application Priority Data
Feb. 14, 2000 (EP) 00102972.7

(51) Int. Cl. A61K 38/00; A61K 31/225

(52) U.S. Cl. 514/2; 514/547

(57) ABSTRACT

Use of compatible solutes for the protection of biopolymers from degradation by degrading enzymes.

The present invention relates to the use of compatible solutes as inhibitors of the enzymatic degradation of macromolecular biopolymers.

DE-A-198 34 816 relates to the use of ectoine or ectoine derivatives in cosmetic formulations. It is disclosed that the mentioned compounds protect and stabilize nucleic acids of the human skin cells from physical, chemical and biological influences, such as radiation, especially ultraviolet radiation, denaturing substances, enzymes, especially endonucleases and restriction enzymes, and viruses, especially herpes viruses.

U.S. Pat. No. 5,039,704 discloses a method of treating a catabolic dysfunction in an animal, wherein a therapeutically effective amount of glutamines or an analogue of glutamine is administered.

U.S. Pat. No. 5,684,045 relates to the treatment of a catabolic gut-associated pathological process, especially intestinal mucosal and pancreatic atrophy, enhanced gut permeability and other diseases. These diseases are treated with a therapeutically effective amount of glutamine or an analogue thereof.

U.S. Pat. No. 5,428,063 relates to a pharmaceutical composition in food supplements for the treatment or prevention of liver diseases. This involves the administration of high doses of betaine.

U.S. Pat. No. 5,827,874 relates to the use of proline for the treatment of inflammations and pain, especially for the treatment of inflammatory conditions, rheumatic and non-rheumatic pain, and for post-surgical and post-traumatic pain.

S. Knapp et al. in Extremophiles (1999), 3(3), 191-8, describe a temperature-stabilizing effect of compatible solutes. Ectoine, hydroxyectoine and betaine are mentioned.

Th. Sauer et al., Biotechnology and Bioengineering (1998), 57 (3), 306-13, discloses a temperature-stabilizing effect of the compatible solutes ectoine, hydroxyectoine and betaine.

EP-A-0 915 167 relates to a method for the in-vivo recovery of components from cells by alternating conditions to which the cells are subjected. Ectoine and hydroxyectoine are described as an effective additive for the cryoprotection of biologically active substances.

The undesired degradation of macromolecular biopolymers is prevented by the specific inhibition of the enzymes catalyzing the reaction.

Surprisingly, it has now been found that the enzymatic degradation of macromolecular biopolymers can also be suppressed by the addition of compatible solutes, without a specific inhibition of the enzymes catalyzing the degradation being necessary.

Therefore, the invention relates to the use of compatible solutes as inhibitors of the enzymatic degradation of macromolecular biopolymers.

Enzymatic Degradation of Biological Macromolecules

Biological macromolecules are synthesized and degraded in, respectively, anabolic and catabolic processes of metabolism. The enzymatic cleavage of organic macromolecules into their monomer compounds may be effected by hydrolysis of phosphorolysis. In hydrolysis, the cleavage is effected with the consumption of water, and in phosphorolysis, with the consumption of phosphate.

The natural catalysts of hydrolysis are hydrolases. Hydrolases include lipases, which cleave fats into glycerol and fatty acids, phospholipases, which are digestive enzymes and cleave ester linkages of phosphatidyl compounds, nucleases, which cleave nucleic acid polymers, such as DNA and RNA, glycosidases, which cleave glycosides, and proteases, which cleave the peptide bonds of proteins.

During proteolysis, i.e., the hydrolysis of proteins, the peptide bonds (also called amide bonds) between the α-amino group of one amino acid and the α-carboxy group of a second amino acid are cleaved with the consumption of a water molecule. In the case of the cleavage of a dipeptide, i.e., a peptide consisting of two amino acids, two free amino acids are formed by the proteolysis.

The proteases known to date are classified into different protease classes or families depending on their manner of functioning and their substrates. The following protease classes have been described:

TABLE 1
Known classes of proteases and typical representatives

| | | Characteristic |
|---|---|---|
| Protease class or | | amino acid group of |
| family | Typical protease | the active site |
| Serine protease 1 | chymotrypsin A, trypsin, | catalytic triad of |
| | elastase, thrombin | aspartate, serine, |
| | | histidine |
| Serine protease 2 | subtilisin | catalytic triad of |
| | | aspartate, serine, |
| | | histidine |
| Cysteine protease | papain, cathepsin B | cysteine, histidine, |
| | | aspartate |
| Aspartate protease | penicillopepsin, renin, | aspartate |
| | pepsin, plasmin | |
| Metallo-protease 1 | carboxypeptidase A, | zinc, calcium, |
| | collagenase | manganese, glutamate, |
| | | tryptophan |
| Metallo-protease 2 | thermolysin | zinc, glutamate, histidine |

Proteolyses can proceed partially (limitedly) or completely (totally). In partial proteolysis, protein fragments or peptides of different sizes are formed, while in total hydrolysis, a protein is completely degraded into amino acids. The protein chains are degraded in a structure-specific or non-specific way from the end of the protein strands by so-called exoproteinases, or after cleavage in the middle of the protein strand by so-called endoproteinases.

Technical Applications of Enzyme-catalyzed Hydrolysis

Enzyme-catalyzed hydrolysis is employed in many technical fields, and in biotechnology, proteolysis is of great importance, in particular. Specific proteases are employed, for example, as biochemical tools for the elucidation of structure/function relationships of proteins. Thus, proteins are subjected to a partial (limited) proteolysis, and it is examined what properties the remaining protein fragments or peptides have. Protease inhibitors are employed in a well-aimed manner for stopping ongoing proteolyses.

Proteolysis is further employed for protein sequence analysis and for peptide mapping. Proteases are also used for analyzing the topology of biological membranes containing proteins and for the solubilization of membrane proteins.

Proteases are also employed on an industrial scale for the catalytic processing of proteins and peptides. Thus, proteases are used in detergents for removing protein contaminations from textiles or for cleaning technical surfaces from protein contaminations. In addition, proteases are utilized for the industrial preparation of peptides or amino acids from proteins.

Protection from Enzymatic Degradation

Biological macromolecules and polymers can be protected from enzymatic degradation.

Thus, for example, proteolysis can be partially or completely prevented by protease-inhibiting substances, so-called protease inhibitors. Protease inhibitors are classified into two classes:

Low-molecular weight inhibitors specifically binding to the active site of a protease which irreversibly modify the amino acid residues in the active site of the proteases in such a way that their functionality is lost.

Protease inhibitors which serve as so-called pseudosubstrates for proteases. Proteases are kind of distracted from their actual protein substrate and stoichiometrically withdrawn from the reaction solution. The proteases, although not destroyed in this case, are specifically removed. Class 2 protease inhibitors are also the type which withdraw cofactors from the enzymes which are essential for their activity.

The former group includes, for example, the serine protease inhibitors diisopropyl phosphofluoridate (DFP) and phenylmethanesulfonyl fluoride (PMSF). Aspartate proteases are inactivated by diazoacetyl compounds and by pepstatin. Metallo-proteases are generally inhibited by metal-chelating reagents. Carboxypeptidases A and B are specifically inhibited by inhibitors which can be isolated from potatoes, and thermolysin is specifically inhibited by phosphoramidon.

The second group of protease inhibitors includes, for example, pancreatic trypsin inhibitor, soybean trypsin inhibitor, α-protease inhibitor, and the universal protease inhibitor α-2-macroglobulin.

G. Salvesen and H. Nagase (1989) give a survey over the class 1 and class 2 inhibitors utilized according to the state of the art [Inhibition of proteolytic enzymes in: Proteolytic enzymes: A practical approach (Editors: R. J. Beynon and J. S. Bond, IRL Press Oxford)].

Due to their principle of action, all class 1 protease inhibitors have the disadvantage that they can basically react with all proteins, not only with proteases or their active sites. Thus, class 1 protease inhibitors have a toxic potential for biological systems and organisms. Class 2 protease inhibitors have one disadvantage, inter alia, in that they only inhibit the proteases stoichiometrically and reversibly. In principle, proteolysis always remains possible.

The previously described methods of protection from enzymatic degradation have in common that the respectively responsible biocatalyst is specifically inhibited.

The proteolysis of proteins can also be prevented by protein denaturing. This involves the complete disruption of the secondary, tertiary and quarternary structures of all proteins, so that specific proteases no longer exhibit any activity. Such denaturing processes are achieved by chaotropic reagents, such as urea or guanidine hydrochloride (guanidinium chloride), or by detergents such as sodium dodecylsulfate. A particular disadvantage in these methods is that although proteolysis is prevented, the proteins to be protected will lose their functions in most cases.

None of the methods stated here aims at a stabilization of the respective substrates.

### DESCRIPTION OF THE INVENTION

Surprisingly, it has been found that the use of compatible solutes prevents the degradation of macromolecular biopolymers, especially of macromolecules, proteins, lipids or nucleic acids, by degrading enzymes.

According to the invention, the substances to be used as compatible solutes are preferably selected from the group consisting of ectoine, derivatives of ectoine, such as hydroxyectoine, proline, betaine, glutamine, cyclic diphosphoglycerate, mannosylglycerate, derivatives of mannosylglycerate, such as mannosylglyceramide, di-myo-inositol phosphate, diglycerol phosphate, N_{γ}-acetylornithine, trimethylamine-N-oxide or combinations thereof.

It is preferred to use the compatible solutes in a concentration of from 0.05 to 2.0 M. When ectoine is used, it is further preferred to use a concentration of from 0.1 to 1 M, especially from 0.1 to 0.5 M. When other solutes are used, from 0.4 to 1.5 M, especially from 0.4 to 1.2 M, is preferred.

According to the invention, a method for the protection of biopolymers from degradation by degrading enzymes is provided in which compatible solutes are added to a sample containing said biopolymers.

The addition of the compatible solute or solutes is optionally followed by an incubation, which is optionally followed by steps of further processing, such as cell lysis and isolation of the degradable biopolymer.

In particular, the sample is a biotechnological starting material for the preparation of proteins or nucleic acids.

The examinations underlying the invention now have surprisingly shown that enzymatic degradation can be prevented by the use of compatible solutes, and strikingly, neither the functionality of the biopolymer to be protected nor that of the degrading enzyme are lost. The results additionally show that macromolecules can be protected from enzymatic attack while small proteins and peptides are not protected.

Thus, when compatible solutes are acting, the degradation seems to be prevented not by the specific inhibition or elimination of the degrading enzymes, but rather through a possible non-specific stabilization of the macromolecular substrates themselves. The addition of compatible solutes seemingly changes the structure of the biopolymer in such a way that the degrading enzyme no longer “recognizes” the biopolymer. Thus, the enzymatic degradation of biomolecules is probably suppressed in a non-specific way by steric enzyme-substrate incompatibility.

Especially for applications with proteins, the application of the invention has the immense advantage that a single inhibition solution becomes universally employable. Frequently, protein solutions contain a broad spectrum of undesirable proteases, which according to the prior art requires a specific inhibitor for each type of protease and requires the use of inhibition mixtures. This approach and the accompanying drawbacks can be circumvented by the method according to the invention.

Connected with the activities described here are also activities of the compatible solutes as medicaments. According to the invention, the compatible solutes can be employed for the preparation of medicaments for the treatment of diseases which are caused by the enzymatic degradation of biopolymers or of structures constituted by biopolymers, such as cells, organelles or tissues, and are causally related to pathological phenomena. These include, in particular, diseases such as diseases of the immune system, such as autoimmune diseases, insulin-dependent diabetes mellitus, Graves disease, Hashimoto's disease, deleterious side-effects from radiation treatments, inflammatory processes, graft rejection, HIV infections or retroviral infections (e.g., herpes), tissue injuries/wound healing, acute and chronic inflammation, acute pancreatitis, shock conditions, fibrinolytic bleeding, heart diseases (e.g., infarction), Alzheimer's disease, neuronal degeneration, diseases of the liver, skeleton and muscles, blood hypertension, metastasis formation of cancer cells, and skin cancer.

### EXAMPLES

The effectiveness of the principle underlying the invention was documented by inhibiting the limited proteolysis of antibodies. The experiments were performed with the following antibodies or conjugates: human IgG: Sigma Product No. 14506 Lot 037H8816; bovine IgG; Serva; Cohn fraction II product No. 22550; monoclonal mouse anti-human IgG: DAKO clone A57H Code No. Mo828 Lot 076 IgM kappa: rabbit anti-mouse IgG+IgM (H+L): Dianova Code No. 315-035-058 Lot 39605.

Limited proteolyses of human IgG were performed at an antibody concentration of 0.5 mg/ml and a pepsin concentration of 5 μg/ml at 37° C. in 100 mM sodium acetate, pH 3. The incubations were performed without additions under 0.5 M ectoine, under 0.5 M hydroxyectoine, and under a mixture of 0.25 M ectoine and 0.25 M hydroxyectoine. The course of the limited proteolyses was quantified subsequent to the incubations in 12% SDS gels under reducing conditions.

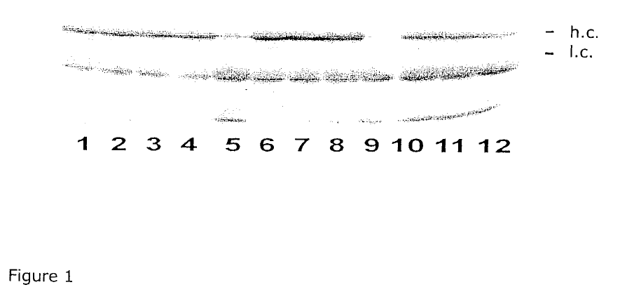

From FIG. 1, it can be seen that the hydrolysis of the heavy chain is inhibited significantly by both ectoine and hydroxyectoine.

FIG. 1 shows the limited proteolysis of the heavy chains (h.c.) of a human antibody (IgG) by pepsin. Lanes 1, 5, 9: with water; lanes 2, 6, 10: with ectoine; lanes 3, 7, 11: with ectoine and hydroxyectoine; lanes 4, 8, 12; with hydroxyectoine; lanes 1, 2, 3, 4: without incubation; lanes 5, 6, 7, 8: after 15 min of incubation; lanes 9, 10, 11, 12: after 180 min of incubation.

The inhibition by ectoine of the proteolysis of antibodies was additionally shown by enzyme-linked immunosorbent assay (ELISA). Thus, 96-well ELISA plates were coated over night with 0.5 μg of human IgG in 100 μl of 0.9% NaCl at 4° C. Subsequently, the plates were washed three times in 50 mM Tris, 0.8% NaCl, 0.020% KCl, pH 7.4, C(T-TBS), and blocked with 200 μl of 1% gelatin in TBS for 2 hours at 37° C. After three washes in T-TBS, an incubation was performed with monoclonal mouse anti-human IgG diluted 1:250 in TBS at 50 μl per well for one hour at 37° C. After four washes with T-TBS, incubation was performed with a peroxidase-conjugated rabbit anti-mouse antibody diluted 1:5000 in TBS at 100 μl per well for one hour at 37° C. After four additional washes, the enzyme reaction was performed by adding 100 μl of substrate per well. As the substrate was used 4.8 mg of phenylene diamine in 3 ml of 0.2 M Na₂HPO₄, 3 ml of 0.1 M citric acid, 6 ml of water, and 5 μl of H₂O₂. The reaction was stopped after about 10 minutes by acidification with 100 μl per well of 0.07 M sulfuric acid, and the plates were quantified in an ELISA autoreader. Prior to the experiment, all three protein components were proteolyzed by pepsin for two hours at 37° C. with or without the addition of 0.5 M ectoine. As can be seen from Table 2, two antibodies could be effectively protected against proteolytic digest by the addition of 0.5 M ectoine. The antibody conjugate was not attacked by pepsin. The treatment was performed in an analogous manner with hydroxyectoine, proline and betaine.

TABLE 2
Residual signal in ELISA after the specified
pretreatment of the individual antibodies
Pretreated antibody

| | | monoclonal | peroxidase |
|---|---|---|---|
| | human IgG | mouse antibody | conjugate |
| without treatment | 1000% | 100% | 100% |
| with ectoine | 85% | 97% | 98% |
| with pepsin | 6% | 59% | 104% |
| with ectoine and pepsin | 91% | 98% | 121% |

The concentration dependence of the effect of the compatible solute was determined using ectoine, hydroxyectoine, proline and betaine as examples. Thus, the human immunoglobulin was proteolyzed as described, and the effect was quantified in ELISA.

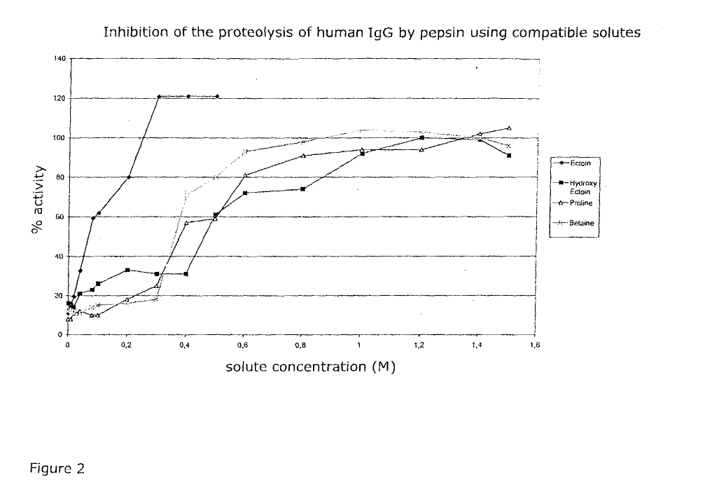

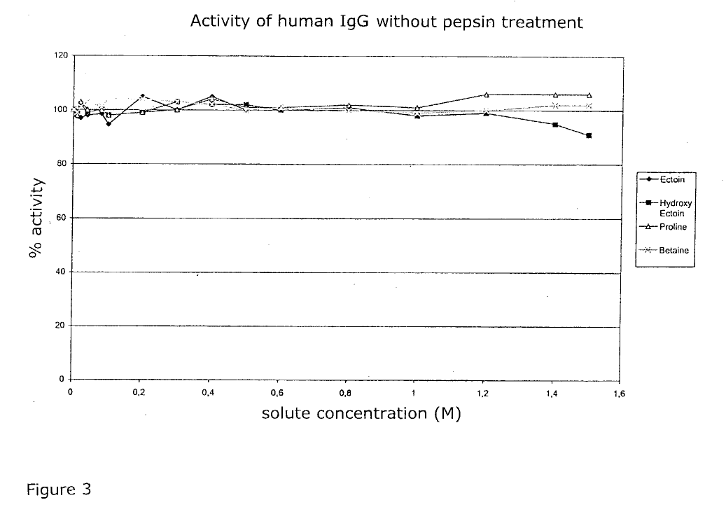

FIG. 2 shows the concentration dependence of the stabilization of a human antibody from proteolysis by pepsin in the presence of increasing concentrations of ectoine, hydroxyectoine, proline and betaine. FIG. 3 shows the result of the control experiments without the addition of pepsin to the mixture of antibody and compatible solute.

In principle, the experiments allow for two possible explanations. On the one hand, the compatible solutes could inhibit the protease. On the other hand, the solute could prevent the attack of the protease according to the invention for steric reasons through stabilization of the native, more compact conformation of the antibody molecule. Therefore, the effect of ectoine and hydroxyectoine on the activity of pepsin was determined by spectrophotometry on the hydrolysis of a synthetic low-molecular weight hexapeptide [E. Schinaith: Clin. Biochem. 22, 91-98 (1989)]. The enzyme-kinetic measurements with Leu-Ser-p-NitroPhe-Nle-Ala-Leu methyl ester as a substrate showed that neither ectoine nor hydroxyectoine inhibits the protease. This is a clear indication of the fact that the inhibition of the antibody proteolyses is to be attributed to steric effects according to the invention.

1. Use of compatible solutes for the protection of biopolymers from degradation by degrading enzymes, wherein said degrading enzymes are selected from the group consisting of proteases, lipases and phosphorylases.

2. The use according to claim 1, wherein said biopolymers are macromolecules, lipids, proteins or fats.

3. The use according to any of claims 1 to 2, wherein substances selected from the group consisting of ectoine, derivatives of ectoine, such as hydroxyectoine, proline, betaine, glutamine, cyclic diphosphoglycerate, mannosylglycerate, mannosylglyceramide, di-myo-inositol 1,1′-phosphate, diglycerol phosphate, N_{γ}-acetylornithin, trimethylamine-N-oxide or combinations thereof are employed as said compatible solutes.

4. The use according to at least one of claims 1 to 3, wherein said compatible solutes are employed in a concentration of from 0.05 to 2 M.

5. A method for the protection of biopolymers from degradation by degrading enzymes selected from the group consisting of proteases, lipases and phosphorylases, wherein compatible solutes are added to a sample containing said biopolymers.

6. The method according to claim 5, wherein the addition of the compatible solute or solutes is followed by an incubation, which is optionally followed by steps of further processing, such as cell lysis and isolation of the degradable biopolymer.

7. The method according to claim 5 and/or 6, wherein the sample is a biotechnological starting material for the preparation of proteins.

8. Use of compatible solutes for the preparation of a medicament for the treatment of diseases, wherein said diseases are selected from the group consisting of insulin-dependent diabetes mellitus, Graves disease, Hashimoto's disease, HIV infections or retroviral infections (e.g., herpes), tissue injuries/wound healing, shock conditions, fibrinolytic bleeding, heart diseases (e.g., infarction), Alzheimer's disease, neuronal degeneration, diseases of the skeleton and muscles, blood hypertension, metastasis formation of cancer cells, and skin cancer.
